(19) United States
(12) Patent Application Publication
(10) Pub. No.: US 2004/0120846 A1
(43) Pub. Date: Jun. 24, 2004

(54) Fungus abatement system

(76) Inventors: Perry C. Bates, Waterville, OH (US); Jim Stehlik, Waterville, OH (US)

Correspondence Address: Thomas N. Young, Young & Basile, P.C., Suite 624, 3001 West Big Beaver Road, Troy, MI, 48084, US

(21) Appl. No.: 10/733,904

(22) Filed: Dec. 11, 2003

Related U.S. Application Data
(60) Provisional application No. 60/435,390, filed on Dec. 20, 2002.
(60) Provisional application No. 60/448,071, filed on Feb. 18, 2003.

(51) Int. Cl. A61L 9/20

(52) U.S. Cl. 422/4; 422/24; 422/121

(57) ABSTRACT

An apparatus and methodology for abating fungi in a building supported on a ground surface and having an upper enclosed living space and a lower enclosed space beneath the upper enclosed space and proximate or beneath the ground. The apparatus includes a blower positioned in the lower enclosed space and having an air inlet and an air exhaust; a plurality of intake conduits having inlet ends adapted to open in the lower enclosed space proximate a lower boundary of that space and outlet ends connected to the inlet of the blower; a plurality of exhaust conduits having inlet ends connected to the exhaust of the blower and outlet ends positioned exteriorly of the building; and a plurality of ultraviolet lamps positioned at spaced locations in the lower enclosed space and establishing germicidal killing zones intercepting and cleansing air moving from the lower enclosed space into the inlet ends of the intake conduits.

### RELATED APPLICATIONS

This application claims the priority of U.S. Provisional Patent Applications Nos. 60/435,390 and 60/448,071, filed on Dec. 20, 2002 and Feb. 18, 2003, respectively.

### BACKGROUND OF THE INVENTION

The present invention relates to fungus abatement and more particularly to a system for use in preventing fungus from forming in a building structure such as a home or an office building.

Fungus is increasingly a problem in homes and office buildings. The fungus typically develops in unconditioned areas of the building such as basements or crawl spaces and is then spread by a natural upward flow of air and/or by the HVAC system to conditioned areas of the building where it contaminates the conditioned areas and generates occupant discomfort and health hazards.

### SUMMARY OF THE INVENTION

The invention provides a method of maintaining a structure free of fungi. According to the invention method, a flow of air is created from an enclosed space within the structure to a location outside of the structure and the flow of air is treated in a germicidal fashion. This basic air handling and treating process results in a continual cleansing of fungi from the enclosed space to preclude contamination of other areas of the structure.

According to a further feature of the invention methodology, the invention is for use with a building situated on a ground surface and having an upper enclosed space and a lower enclosed space beneath the upper enclosed space and proximate or beneath the ground surface and the method comprises the steps of creating the flow of air from the lower enclosed space to a location outside of the building. creating the flow of air from the lower enclosed space to a location outside of the building.

According to a further feature of the invention methodology, the invention is for use with a structure having a first enclosed space intended for human occupancy and a second enclosed space proximate the first enclosed space and the method comprises creating a flow of air from the second enclosed space to a location outside of the building and treating the flow of air in a germicidal fashion. This basic air handing and treating process results in a continual cleansing of fungi from the second enclosed space to preclude contamination of the first enclosed space by the fungi.

According to a further feature of the invention methodology, the treating step comprises creating a fungi killing zone in the second enclosed space and passing the flow of air through the killing zone. This methodology insures that all air being evacuated from the second enclosed space is passed through a killing zone to remove the fungi from the air.

According to a further feature of the invention methodology, the killing zone comprises a zone in which the flow of air is subjected to radiant energy. This methodology provides a convenient means of creating the killing zone to destroy the fungi. In the disclosed embodiment of the invention the radiant energy comprises ultraviolet radiation.

According to a further feature of the invention methodology, the second enclosed space comprises a finished basement area of the structure including paneling spaced from a foundation wall of the basement to define a dead air space between the foundation wall and the paneling; the fungi killing zone is created in the dead air space; and the flow of air is created from the dead air space to the outside of the structure.

The invention also provides an apparatus for abating fungi in a structure having boundary walls defining a first enclosed space intended for human occupancy and a second enclosed space proximate the first enclosed space. The abatement apparatus comprises a blower unit having an air inlet and an air exhaust and adapted to be positioned within the structure with the air inlet communicating with the second enclosed space and the air exhaust communicating with the exterior of the structure, actuation of the blower unit being operative to draw air from the lower enclosed space into the blower unit inlet and thereafter through the exhaust conduit to the exterior of the building; and a source of radiant energy adapted to be positioned in the second enclosed space in a position to intercept the air moving from the second enclosed space into the inlet of the blower unit. This apparatus provides a ready and continuous cleansing of the air in the second enclosed space and insures that all of the exhausted air is treated with radiant energy to remove the fungi from the air.

According to a further feature of the invention apparatus, the second enclosed space comprises a lower enclosed space in the form of a finished basement area of a building structure including paneling spaced from a foundation wall of the basement to define a dead air space between the foundation wall and the paneling; the fungi killing zone is established in the dead air space; and the intercepted air comprises air moving from the dead air space into the inlet of the blower unit.

According to a further feature of the invention apparatus, an intake conduit is provided including a horizontal run connected to the blower unit air inlet and a vertical run extending downwardly from the horizontal run to position the inlet end of the intake conduit proximate a floor surface of the lower enclosed space. This arrangement insures an effective and continual evacuation of the air in the lower enclosed space and facilitates movement of the air through the radiant energy zone. In the disclosed embodiment, a plurality of spaced intake conduits are provided each having a horizontal run connected to the blower unit air inlet and a vertical run defining an air inlet positioned proximate the floor surface of the lower enclosed space.

According to a further feature of the invention apparatus, the source of radiant energy comprises a plurality of radiant energy sources adapted to be positioned in spaced relation in the lower enclosed space and operative to intercept the air moving into the intake end of each of the intake conduits. This arrangement insures that substantially all of the air will be treated by radiant energy for fungus removal before entering the exhaust system.

According to a further feature of the invention apparatus, each source of radiant energy comprises a source of ultraviolet radiation in the form of an ultraviolet lamp. This arrangement provides a ready and efficient means of providing the desired germicidal effect.

According to a further feature of the invention apparatus, the apparatus further includes means for sensing the humidity in the lower enclosed space and operative to actuate the blower unit and the ultraviolet lamps in response to variations in the sensed humidity.

The invention also provides a building structure including boundary walls defining a first enclosed space intended for human occupancy and a second enclosed space proximate the first enclosed space; a blower unit positioned in the structure and having an air inlet communicating with the second enclosed space and an air exhaust communicating with the exterior of the building structure, actuation of the blower unit being operative to draw air from the second enclosed space into the inlet of the blower unit and thereafter discharge the air through the air exhaust to the exterior of the building structure; and a source of radiant energy positioned in the second enclosed space in a position to establish a fungi killing zone to intercept air moving from the second enclosed space into the inlet of the blower unit.

According to a further feature of the invention, the first enclosed space comprises an upper enclosed space including a floor; the second enclosed space comprises a lower enclosed space positioned beneath the floor and including a lower boundary surface; the blower unit is positioned in the lower enclosed space beneath the floor; at least one intake conduit is provided having an inlet end opening in the lower enclosed space and an outlet end connected to the blower end air unit; at least one exhaust conduit is provided having an inlet end connected to the air exhaust of the blower unit and an outlet end communicating with the exterior end of the building, whereby actuation of the blower unit is operative to draw air from the lower enclosed space through the inlet end of the intake conduit to the blower unit and thereafter through the exhaust conduit to the exterior of the building; and the source of radiant energy is positioned in the lower enclosed space in a position to intercept the air moving from the lower enclosed space into the inlet end of the intake conduit. This building construction insures that fungal matter forming in the lower enclosed space will not contaminate the upper enclosed space.

According to a further feature of the invention, the lower enclosed space comprises a basement; the building structure includes paneling spaced from a foundation wall of the basement to define a dead air space between the foundation wall and the paneling; the fungi killing zone is established in the dead air space; and the intercepted air comprises air moving from the dead air space into the inlet end of the intake conduit.

According to a further feature of the invention, the inlet end of the intake conduit is positioned proximate the lower boundary surface of the lower enclosed space. This arrangement provides a ready and efficient evacuation of air from the lower enclosed space.

According to a further feature of the invention, the source of radiant energy is positioned proximate the floor of the upper enclosed space. This arrangement provides a convenient means of allowing the radiant energy to access the air moving toward the inlet end of the intake conduit.

According to a further feature of the invention, there are a plurality of intake conduits each defining an inlet end opening in the lower enclosed space at spaced locations within the lower enclosed space. This arrangement insures that all of the air in the lower enclosed space will be continually evacuated.

According to a further feature of the invention, there are a plurality of sources of radiant energy positioned proximate the floor in spaced relation within the lower enclosed space and operative, cumulatively, to intercept substantially all of the air moving from the lower enclosed space into the inlet ends of the intake conduits. This arrangement insures that all of the air leaving the lower enclosed space will be provided with a germicidal treatment. In the disclosed embodiment of the invention, each source of radiant energy comprises a source of ultraviolet energy in the form of an ultraviolet lamp.

Other applications of the present invention will become apparent to those skilled in the art when the following description of the best mode contemplated for practicing the invention is read in conjunction with the accompanying drawings.

### BRIEF DESCRIPTION OF THE DRAWINGS

The description herein makes reference to the accompanying drawings wherein like reference numerals refer to like parts throughout the several views, and wherein:

FIG. 1 is a perspective, fragmentary view of building having a crawl space employing a fungus abatement system according to the invention;

FIGS. 2, 3, 4 and 5 are perspective, plan, side elevation, and end views of a blower unit employed in the fungus abatement system;

FIG. 6 is a plan view of the fungus abatement system;

FIGS. 7 and 8 are cross-sectional views of germicidal lamp assemblies utilized in the fungus abatement system;

FIG. 9 is a wiring diagram for a fungus abatement system according to the invention;

FIG. 10 is a perspective view showing the fungus abatement system of the invention utilized in a building having a full unfinished basement; and

FIGS. 11-13 are fragmentary views showing the fungus abatement system of the invention utilized in a building having a full finished basement.

### DETAILED DESCRIPTION OF THE PREFERRED EMBODIMENTS

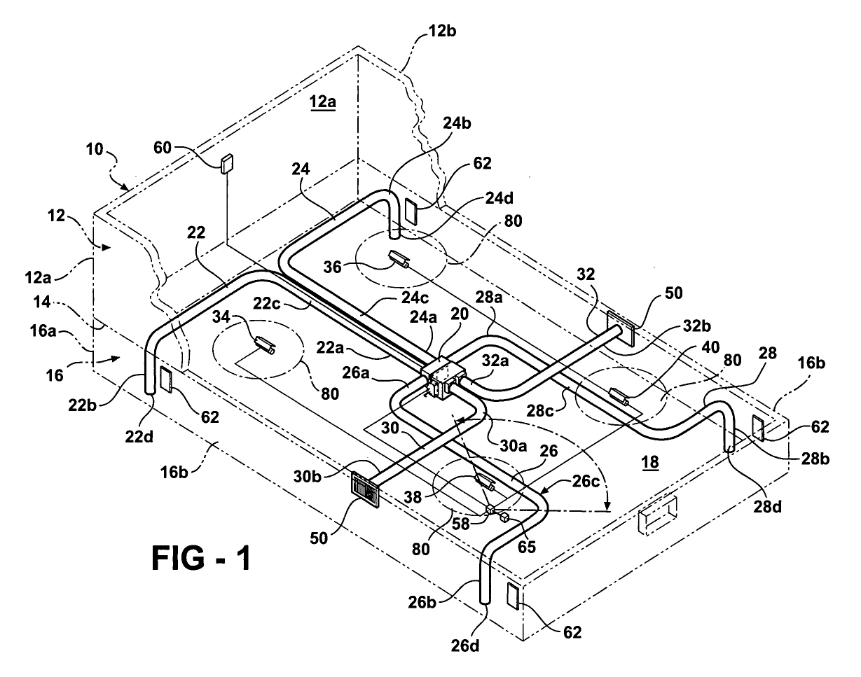

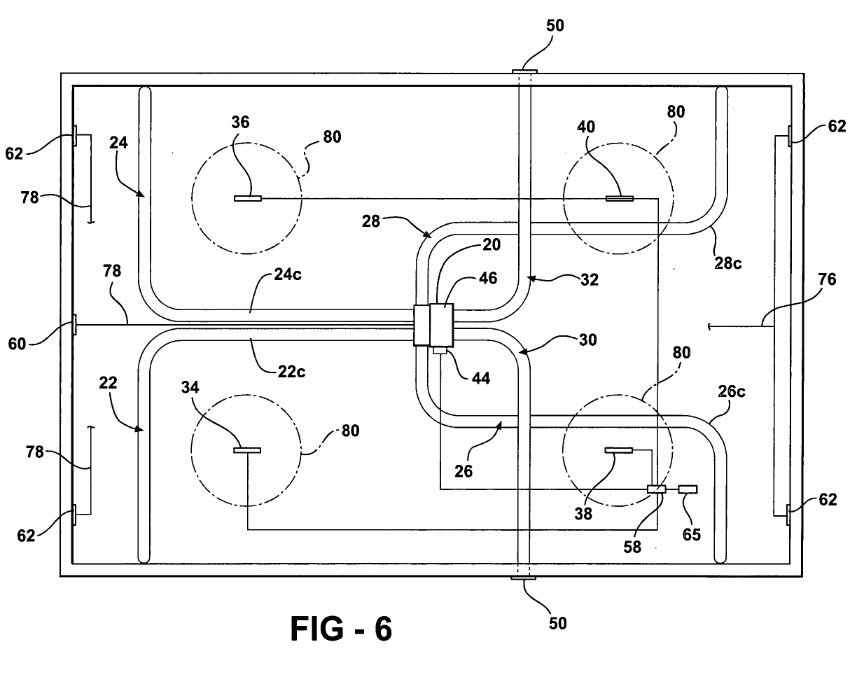

The invention fungus abatement system is seen in FIGS. 1 and 6 installed in a building 10 of the type including an upper enclosed living area space 12, bounded by a floor 14, and a lower enclosed crawl space 16 beneath the upper enclosed space. Upper enclosed space 12 is defined by walls 12a and 12b as well as by floor 14 and crawl space 16 is defined by walls 16a and 16b as well as by a ground surface 18.

The fungus abatement system, broadly considered, includes a blower assembly 20, a plurality of air inlet conduits 22, 24, 26 and 28, a pair of exhaust conduits 30 and 32, and a plurality of germicidal units 34, 36, 38 and 40.

Blower assembly 20 includes a housing 42 and a pair of squirrel cage blowers 44 and 46.

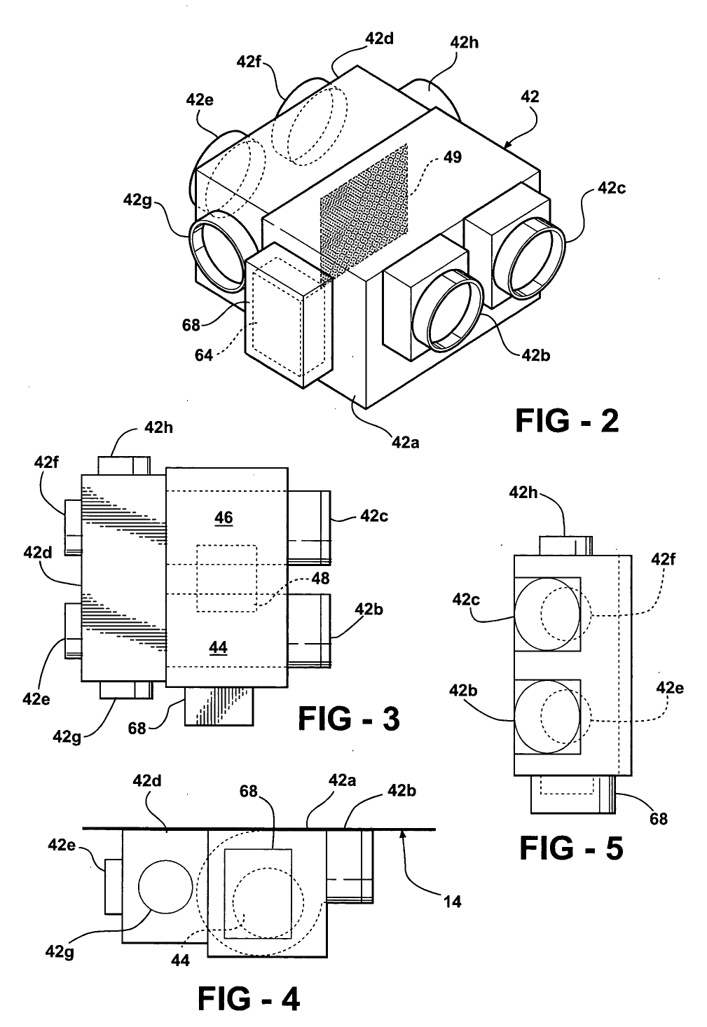

Housing 42 (FIGS. 2-5) has a sheet metal construction and is secured to the underface of floor 14 centrally within the crawl space 16. Housing 42 includes a main body portion 42a defining exhaust ports 42b and 42c, and a plenum chamber 42d positioned against main body portion 42a and defining intake ports 42e, 42f, 42g and 42h.

Squirrel cage blowers 44 and 46 are commonly driven by a central electric motor 48 positioned in housing main body portion 42a and may each comprise a unit available from Penn Zepher as Part Number Z102. Each blower 44, 46, will be understood to have an exhaust communicating with a respective exhaust port 42b, 42c and an intake communicating with plenum chamber 42d. Blower assembly 20 is preferably provided with a germicidal filter 49 positioned at the interface of plenum chamber 42d and the intakes of the blowers 44 and 46.

Intake conduits 22, 24, 26 and 28 each have an outlet end 22a, 24a, 26a, and 28a connected respectively to a housing port 42e, 42f, 42g and 42h; an inlet end 22b, 24b, 26b and 28b positioned respectively in the four corners of the crawl space; and an intermediate portion 22c, 24c, 26c and 28c interconnecting the inlet end and the outlet end of each conduit. Inlet ends 22b, 24b, 26b and 28b will be seen to be vertically disposed and will be seen to terminate in an inlet port 22d, 24d, 26d and 28d positioned proximate but spaced slightly above the ground surface 18. Intermediate portions 22c, 24c, 26c and 28c will be seen to comprise horizontal runs extending beneath floor 14 and interconnecting the respective inlet end and the respective outlet end of the respective conduit.

Exhaust conduits 30, 32 each define an inlet end 30a and 32a connected to a respective port 42b, 42c of housing 42 and an outlet end 30b and 32b communicating with a register or vent 50 positioned in opposite crawl space sidewalls 16b.

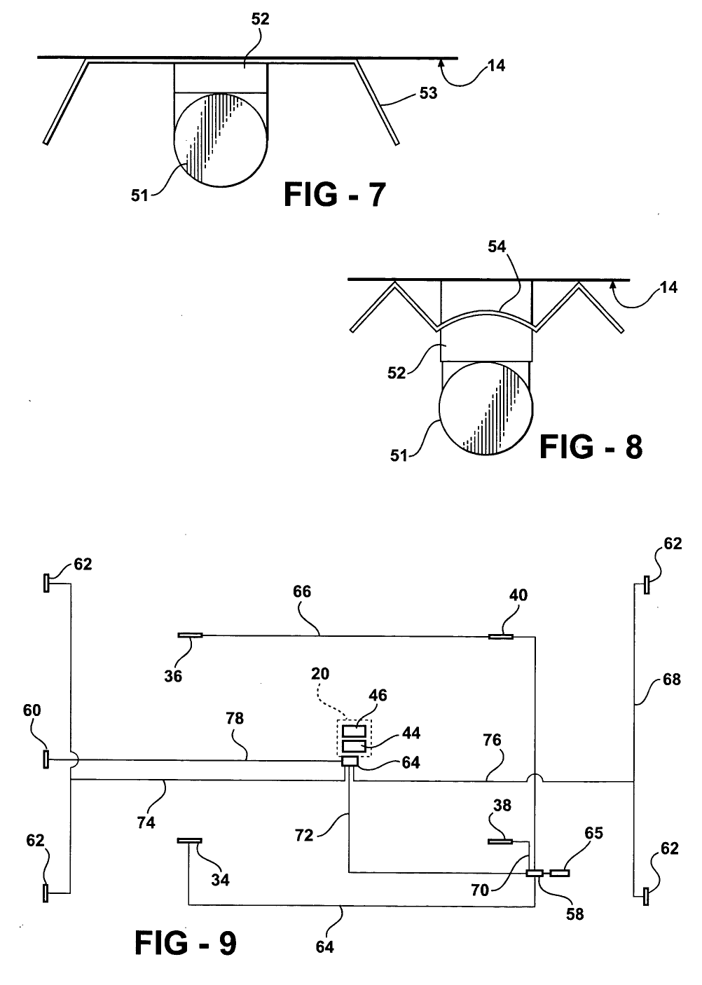

Germicidal units 34, 36, 38 and 40 are positioned on the underface of floor 14 in association with the inlet port of a respective intake conduit. Each germicidal unit may comprise, for example, a 15 watt ultraviolet germicidal lamp of the type available from Sylvania company as Part Number G15T8. Each germicidal lamp in known manner emits ultraviolet radiation in the wave length of 254 nm which has the effect of establishing a killing zone around each germicidal unit which will effectively kill any fungi carried by air passing through the killing zone. Each germicidal lamp comprises an elongated tube 51 and a base 52 to which the tube is suitably mounted. If desired, an overhead directional reflector may be provided with respect to at least certain of the lamps. The reflector may, for example, have an inverted trough configuration as seen at 53 in FIG. 7 or a gull wing configuration as seen at 54 in FIG. 8, depending upon the shape and size of the killing field that it is desired to establish in the vicinity of the tube 51. Preferably, however, no reflectors would be utilized in the crawl space embodiment of FIGS. 1-6. Rather, sufficient germicidal lamps would be provided to essentially flood the crawl space area with radiant energy.

The fungus abatement system of the invention further includes a motion detector 58, a control panel 60, a plurality of humidistats 62, and a controller 64.

Motion detector 58 may be installed in the crawl space 16 beneath the floor 14 and preferably has a 180° sweep. The detector may be of the type available from Desa International as Part Number 5411-ASL-5407A. This is a motion-on detector and is therefore used with a relay 65 to reverse the action of the motion detector to a motion-off detector. Relay 65 may be a 5 pin 6C895-7 type and may snap into a 5 pin base of the 6C898-1 type.

Control panel 60 may be positioned in upper enclosed living space 12 on wall 12a for ready access by occupants of living space 12.

A humidistat or humidistat trigger 62 may be installed in crawl space 16 proximate the inlet port 22d, 24d, 26d and 28d of each of the intake conduits whereby to sense the humidity of the air entering each of the intake conduits.

Controller 64 may be mounted, for example, in a controller housing 68 secured to a side face of blower housing 42.

As seen in the wiring diagram of FIG. 9, a lead 64 interconnects lamp 34 and motion detector 58; a lead 66 interconnects lamp 36 and lamp 40; a lead 68 interconnects lamp 40 and motion detector 60; a lead 70 interconnects lamp 38 and motion detector 60; a lead 72 interconnects motion detector 60 and controller 64; leads 74 and 76 interconnect thermostats 62 and controller 64; and a lead 78 interconnects control panel 60 and controller 64.

### Operation

With control panel 60 calling for operation of the fungus abatement system, and assuming that the motion detector 58 does not detect the presence of anyone in the crawl space, the controller 64 functions to turn on the system and specifically functions to turn on the blowers 44, 46 and the lamps 34, 36, 38 and 40. Actuation of the blowers has the effect of drawing air from the crawl space 16 into the inlet ports 22d, 24d, 26d and 28d of the intake conduits for passage through the conduits to the plenum chamber 42d and thence through the squirrel cage blowers for discharge via the conduits 30 and 32 through the grills 50 to the exterior of the building. As the air moves respectively toward the inlet ports 22d, 24d, 26d and 28d of the intake conduits, the air passes through killing zones 80 established around each of the lamps 34, 36, 38, 40 so that effectively all of the air entering the inlet ports 22d of all of the conduits is first passed through a killing zone where the air is irradiated by the germinating lamp to kill any fungus or other contaminants carried by the air. The air passing through the intake conduits in turn passes through germicidal filter 49. The air thereafter moved outwardly through the exhaust conduits is thus essentially free of fungus and the air in the crawl space 16 is continuously purged of fungus so that the crawl space air, rather than rising upwardly laden with fungal contaminants into the conditioned air living area space above the crawl space, is cleansed within the crawl space and carried to a location outside of the building. Alternatively, the system may be programmed to cycle on and off dependent upon the readings provided by the humidistats 62. Specifically, as the humidity of one or more of the humidistats reaches a predetermined upper limit the controller functions to turn on the system and as the humidity reaches a predetermined lower limit as determined by the humidistats the blowers are turned off. Desirably, the ultraviolet lights remain on for a measured period of time following cessation of blower operation to insure that the stagnant air remaining in the crawl space is cleansed of fungi.

It will be understood that, depending upon the construction and porosity of the building, air will also be sucked downwardly from the conditioned air space 12 into the crawl space for discharge through the intake conduits and the exhaust conduits to the exterior of the building, thereby reversing the normal flow of air within the building.

It will further be understood that the efficiency of ultraviolet radiation is directly proportional to the density or the humidity of the air being treated. The denser or more humid the air, the slower the ultraviolet travel. Accordingly, by lowering humidity the efficiency of the germicidal units increases. In some scenarios involving exceptionally high humidity, it may be necessary to provide a separate dedicated dehumidifier to assist the invention system in maintaining a desired humidity level.

It will further be understood that, if the motion detector 58 detects movement in the crawlspace, the controller is appropriately signaled to turn off the system to preclude harm to living creatures in the crawlspace.

### Alternate Embodiments

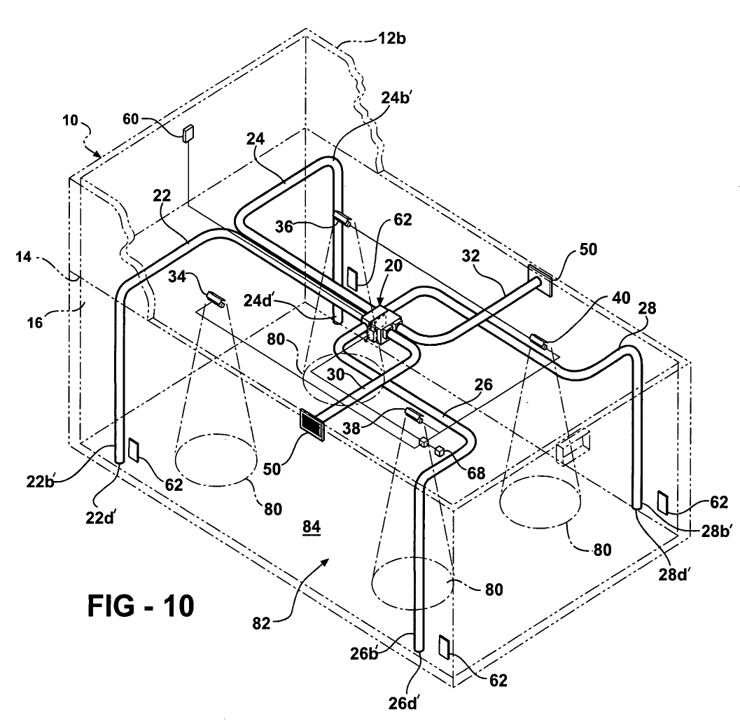

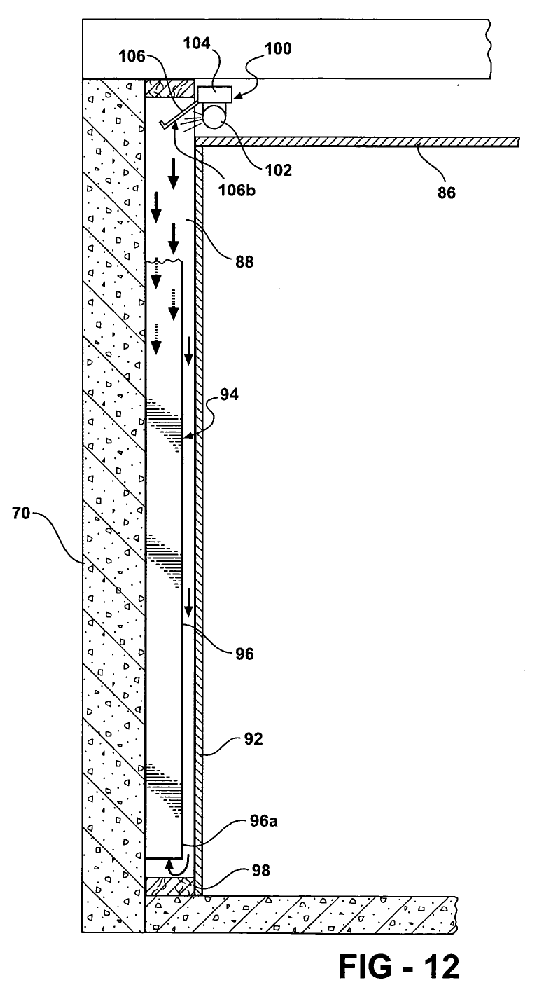

The fungus abatement system seen in FIG. 10 is intended for use with a building 10′ having a full unfinished basement 82 including a floor 84. The system of FIG. 12, for use with a full unfinished basement, is identical to the system of FIG. 1, for use with a crawl space, except that the intake conduit lower ends 22b′, 24b′, 26b′, and 28b′ are extended vertically downwardly to position the conduit inlet ports 22d′, 24d′, 26d′ and 28d′ proximate the floor 84, and the humidistats 62 are moved downwardly to retain their positions proximate the inlet ports of the respective intake conduits whereby to monitor the humidity of the air entering the respective conduits. As with the crawl space configuration, sufficient germicidal lamps would be provided to essentially flood the basement area with radiant energy or, alternatively, at least certain of the ultraviolet lamps would be provided with directional reflectors. Lamps 34, 36, 38 and 40 are preferably mounted on the underface of floor 14.

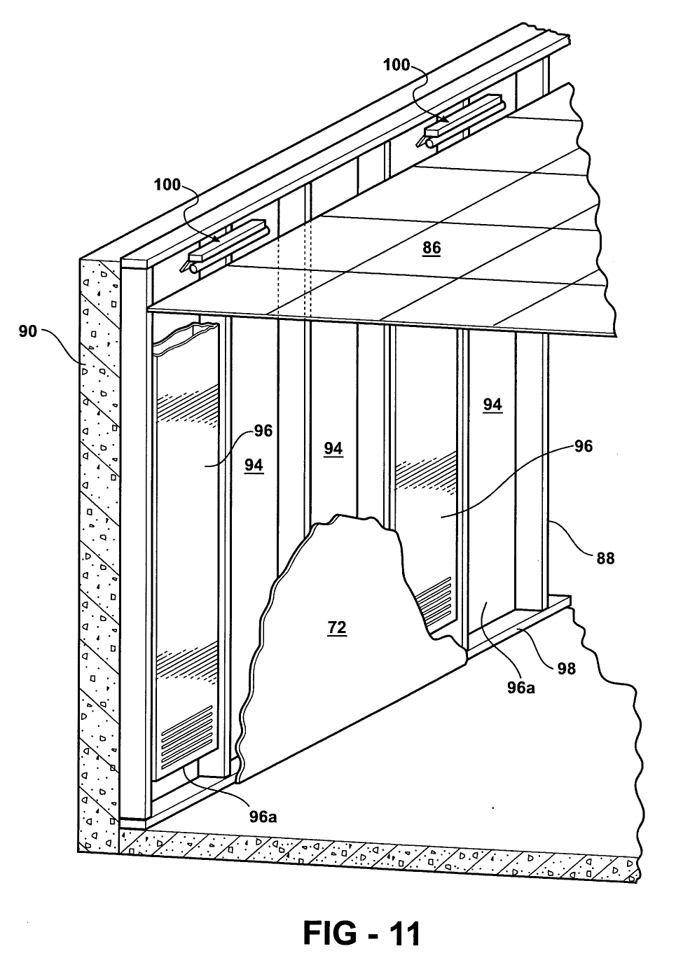

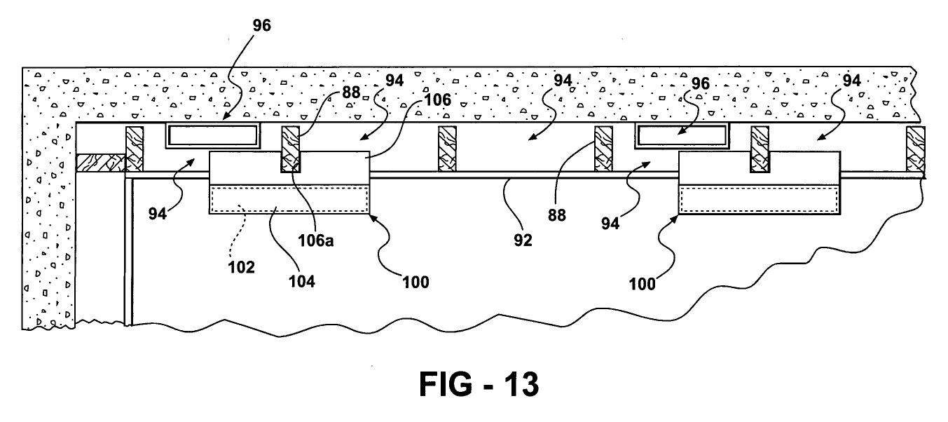

FIGS. 11-13 illustrate an arrangement for use in a full finished basement including a drop ceiling 86, studs 88 mounted against foundation wall 90, and dry wall or other paneling 92 mounted on the studs and defining dead air spaces 94 between the paneling and the foundation wall. Suitable HVAC equipment is provided so that the lower area within the paneling is provided year round with conditioned air, either heated or cooled. The fungus abatement system for the full finished basement of FIGS. 11-13 includes a plurality of vertical intake conduits 96 positioned between selected studs 88 with the open lower ends 96a spaced above the sills 98 and a plurality of germicidal lamp units 100 positioned above the drop ceiling proximate to the perimeter of the basement. For example, and as shown, intake conduits 96 may be positioned around the perimeter of the basement on 48″ centers and a germicidal lamp 100 may be provided in association with each intake conduit. Each germicidal lamp 100 may include an elongated tube 102, a base 104, and a reflector 106. Each lamp may be centered on a stud 88 and the reflector 106 may be notched at 106a to fit over the stud. Each reflector 100 may be of the type available from Simkar Corporation as Part Number ARW20-SR and will be seen to provide an angled reflector surface 106b which is operative to direct rays from the tube 102 downwardly into the dead air spaces 94 on either side of the stud over which the reflector is fitted so as to establish germicidal killing zones in the dead air spaces on either side of the stud over which the reflector is fitted.

It will be understood that the blower unit 20 in this finished basement embodiment is positioned centrally above the drop ceiling, that each conduit 96 is suitably connected to the intake of the blower unit, and that suitable humidistats (not shown) might be provided proximate the intake of the various conduits 96. In operation, following actuation of the blower unit and the germicidal lamps, any fungal matter in the dead air spaces 94 is killed by exposure to the ultraviolet killing zones established in the dead air spaces and the cleansed air is sucked upwardly through conduits 96 for discharge by the blower unit outside of the building. Since the studs 88 do not sealingly interface with the foundation wall but rather define significant spacing at the interface, air is free to move laterally from the dead air spaces in which a conduit is not positioned into a dead air space in which a conduit is positioned for entry into that conduit and discharge from the building. As the air moves laterally toward the intake of a conduit, it moves through a killing zone and is cleansed of fungal matter.

### Specifications

The number sizing and location of the various components of the mold abatement system will of course depend on whether a crawl space is being treated or a full basement is being treated and will of course in each case further depend on the size of the crawl space or the full basement.

As an example, for a crawl space with dimensions of 26′ wide by 42′ long and 36″ deep for a total of 3,276 cubic feet, the blower assembly 20 would have a 638 cfm capacity and would serve to establish a system static pressure of 0.375 inches, and would operate on 3.6 amps. This arrangement would serve to change the air within the crawl space ten times per hour. As previously noted, blowers 44 and 46 in this crawl space configuration may comprise units available from Penn Zepher as Part Number Z102. These blower units would also be satisfactory for use in the full finished basement embodiment of FIGS. 11-13.

As a further example, for a full unfinished basement 8′ deep by 26′ wide by 42′ long, resulting in 8,736 cubic feet of space, a 950 cfm blower assembly 20 would be required operating at 0.8375 inches system static pressure. This arrangement would serve to change the air within the basement 5.868 times per hour. Blowers 44 and 46 in this full basement configuration may comprise units available from Penn Zepher as Part Number Z121.

The invention would seem to provide an efficient and inexpensive means of precluding the contamination of the living areas of a building by fungi.

While the invention has been described in connection with what is presently considered to be the most practical and preferred embodiments, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law. For example, the term fungi as used in the specification and appended claims will be understood to include germs, parasites, spores, bacteria, mold, rust, mildew, smuts, mushrooms and other airborne contaminants. As a further example, the particular reflector configuration, if any, employed in association with the ultraviolet lamps will vary depending upon the nature and configuration of the space being treated. As a yet further example, although the invention has been described with reference to the germicidal treatment of air in a lower enclosed space of a building, it also has applicability in certain situations to the germicidal treatment of air in an upper enclosed space of a building. As a yet further example, although the invention has been described with reference to treatment of air in a building, it may also have applicability to the treatment of air in structures other than buildings.

What is claimed is:

1. A method of maintaining a building structure free of fungi comprising the steps of:
creating a flow of air from an enclosed space within the structure to a location outside of the structure; and
treating the flow of air in a germicidal fashion.

2. A method according to claim 1 wherein the treating step comprises:
creating a fungi killing zone in the lower enclosed space; and
passing the flow of air through the killing zone.

3. A method according to claim 2 wherein the killing zone comprises a zone in which the flow of air is subjected to radiant energy.

4. A method according to claim 3 wherein the radiant energy comprises ultraviolet radiation.

5. For use with a building structure situated on a ground surface and having an upper enclosed space and a lower enclosed space beneath the upper enclosed space and proximate or beneath the ground surface, a method of maintaining the building free of fungi comprising the steps of:
creating a flow of air from the lower enclosed space to a location outside of the building; and
treating the flow of air in a germicidal fashion.

6. A method according to claim 5 wherein the treating step comprises:
creating a fungi killing zone in the lower enclosed space; and
passing the flow of air through the killing zone.

7. A method according to claim 6 wherein the killing zone comprises a zone in which the flow of air is subjected to radiant energy.

8. A method according to claim 7 wherein the radiant energy comprises ultraviolet radiation.

9. A method according to claim 8 wherein:
the lower enclosed space comprises a finished basement area of the building including paneling spaced from a foundation wall of the basement to define a dead air space between the foundation wall and the paneling;
the fungi killing zone is created in the dead air space; and
the flow of air is created from the dead air space to the outside of the building.

10. For use with a structure having a first enclosed space intended for human occupancy and a second enclosed space proximate the first space, a method of maintaining the structure free of fungi comprising the steps of:
creating a flow of air from the second enclosed space to a location outside of the structure;
creating a zone of radiant energy in the second enclosed space; and
passing the flow of air through the radiant energy zone.

11. A method according to claim 10 wherein the radiant energy is in the form of wave energy.

12. A method according to claim 11 wherein the radiant energy is in the form of ultraviolet waves.

13. A method according to claim 10 wherein the method includes the further steps of providing a means for detecting the presence of a human in the second enclosed space and extinguishing the radiant energy in response to a sensed human presence.

14. A method according to claim 13 wherein the method includes the further step of providing the first enclosed space with relatively conditioned air.

15. An apparatus for abating fungi in a structure having boundary walls defining a first enclosed space intended for human occupancy and a second enclosed space proximate the first enclosed space, the apparatus comprising:
a blower unit having an air inlet and an air exhaust and adapted to be positioned in the structure with the air inlet communicating with the second enclosed space and the air exhaust communicating with the exterior of the structure, actuation of the blower unit being operative to draw air from the second enclosed space into the inlet of the blower unit and thereafter discharge the air through the air exhaust to the exterior of the structure; and
a source of radiant energy adapted to be positioned in the second enclosed space in a position to establish a fungi killing zone to intercept air moving from the second enclosed space into the inlet of the blower unit.

16. A structure according to claim 15 wherein the source of radiant energy comprises an ultraviolet lamp.

17. An apparatus according to claim 16 wherein the apparatus further includes an exhaust conduit having an inlet end connected to the exhaust of the blower unit and an outlet end adapted to be positioned at a location outside of the structure.

18. An apparatus for abating fungi in a building supported on a ground surface and having an upper enclosed space and a lower enclosed space beneath the upper enclosed space and proximate or beneath the ground surface, the apparatus comprising;
a blower unit having an air inlet and an air exhaust;
at least one exhaust conduit having an inlet end connected to the exhaust of the blower unit and an outlet end adapted to be positioned at a location outside of the building structure, actuation of the blower unit being operative to draw air from the lower enclosed space into the inlet of the blower unit and thereafter through the exhaust conduit to the exterior of the building structure; and
a source of radiant energy adapted to be positioned in the lower enclosed space in a position to establish a fungi killing zone to intercept air moving from the lower enclosed space into the inlet of the blower unit.

19. An apparatus according to claim 18 wherein:
the lower enclosed space comprises a finished basement area of the building including paneling spaced from a foundation wall of the basement to define a dead air space between the foundation wall and the paneling;
the fungi killing zone is established in the dead air space; and
the intercepted air comprises air moving from the dead air space into the inlet of the blower unit.

20. An apparatus according to claim 18 wherein the apparatus further includes an intake conduit having a horizontal run connected to the blower unit air inlet and a vertical run extending downwardly from the horizontal run to position the inlet end of the intake conduit proximate the floor surface of the lower enclosed space.

21. An apparatus according to claim 20 wherein a plurality of spaced intake conduits are provided each having a horizontal run connected to the blower unit air inlet and a vertical run defining an air inlet end positioned proximate the floor surface of the lower enclosed space.

22. An apparatus according to claim 21 wherein the source of radiant energy comprises a plurality of radiant energy sources adapted to be positioned in spaced relation in the lower enclosed space and operative to intercept the air moving into the intake ends of each of the intake conduits.

23. An apparatus according to the claim 22 wherein each source of radiant energy comprises a source of ultraviolet radiation.

24. An apparatus according to claim 23 wherein each source of ultraviolet radiation comprises an ultraviolet lamp.

25. An apparatus according to claim 24 wherein the apparatus further includes means for sensing the humidity in the lower enclosed space and operative to actuate the blower unit and the ultraviolet lamps in response to variations in the sensed humidity.

26. An apparatus according to claim 25 wherein the apparatus further includes means for detecting the presence of a human in the lower enclosed space and operative in response to such detection to turn off the lamps.

27. An apparatus according to claim 26 wherein the means for detecting the presence of a human comprises a motion detector.

28. A structure comprising:
boundary walls defining a first enclosed air space intended for human occupancy and a second enclosed air space proximate the first air space;
a blower unit positioned in the structure and having an air inlet communicating with the second enclosed space and an air exhaust communicating with the exterior of the structure, actuation of the blower being operative to draw air from the second enclosed space into the inlet of the blower unit and thereafter discharge the air through the air exhaust to the exterior of the building structure; and
a source of radiant energy positioned in the second enclosed space in a position to establish a fungi killing zone to intercept air moving from the second enclosed air space into the inlet end of the blower unit.

29. A structure according to claim 28 wherein the building structure further includes:
an intake conduit having an inlet end opening in the enclosed space and an outlet end connected to the blower unit, whereby the air moving from the enclosed space into the blower unit moves through the intake conduit; and
an exhaust conduit having an inlet end connected to the blower unit air exhaust and an outlet end communicating with the exterior of the building structure where by the air leaving the blower air exhaust moves through the exhaust conduit to the exterior of the building structure.

30. A structure according to claim 28 wherein the source of radiant energy comprises an ultraviolet lamp.

31. A structure according to claim 28 wherein the structure further includes means for providing conditioned air to the first enclosed space.

32. A building structure including:
boundary walls defining an upper enclosed space including a floor and a lower enclosed space defined beneath the floor and including a lower boundary surface;
a blower unit positioned beneath the floor and having an air inlet and an air exhaust;
at least one exhaust conduit having an inlet end connected to the air exhaust of the blower unit and an outlet end communicating with the exterior of the building, actuation of the blower unit being operative to draw air from the lower enclosed space into the air inlet of the blower unit and thereafter through the exhaust conduit to the exterior of the building structure; and
a source of radiant energy positioned in the lower enclosed space in a position to establish a fungi killing zone to intercept air moving from the lower enclosed space into the inlet of the blower unit.

33. A building structure according to claim 32 wherein:
the lower enclosed space comprises a finished basement of the building structure including paneling spaced from a foundation wall of the basement to define a dead air space between the foundation wall and the paneling;
the fungi killing zone is established in the dead air space; and
the intercepted air comprises air moving from the dead air space into the air inlet of the blower unit.

34. A building according to claim 33 wherein the building further includes an intake conduit having an outlet end connected to the blower unit air inlet and an inlet end positioned proximate the lower boundary surface of the lower enclosed space.

35. A building according to claim 33 wherein the blower unit is positioned proximate the floor of the upper enclosed space.

36. A building according to claim 35 wherein there are a plurality of intake conduits each defining an inlet end opening in the lower enclosed space at spaced locations within the lower enclosed space.

37. A building according to claim 36 wherein there are a plurality of sources of radiant energy positioned proximate the floor in spaced relation within the lower enclosed space and operative, cumulatively, to intercept substantially all of the air moving from the lower enclosed space into the inlet ends of the intake conduits.

38. A building according to claim 37 wherein each source of radiant energy comprises a source of ultraviolet energy.

39. A building according to claim 38 wherein each source of ultraviolet energy comprises an ultraviolet lamp.
